(19) United States
(12) Patent Application Publication
(10) Pub. No.: US 2022/0346709 A1
(43) Pub. Date: Nov. 3, 2022

(54) SYSTEM FOR MONITORING OF THE FUNCTIONAL STATUS OF IMPLANTED HEART VALVES

(71) Applicants: THE JOHNS HOPKINS UNIVERSITY, Baltimore, MD (US); Ohio State Innovation Foundation, Columbus, OH (US)

(72) Inventors: Rajat Mittal, Vienna, VA (US); Jung Hee Seo, Baltimore, MD (US); Lakshmi Prasad Dasi, Dublin, OH (US); Hoda Hatoum, Columbus, OH (US); Shantanu Bailoor, Baltimore, MD (US)

(21) Appl. No.: 17/621,531

(22) PCT Filed: Jun. 22, 2020
(86) PCT No.: PCT/US2020/038922

§ 371 Date: Dec. 21, 2021

Related U.S. Application Data
(60) Provisional application No. 62/864,871, filed on Jun. 21, 2019.

(51) Int. Cl. A61B 5/00; A61F 2/24

(52) U.S. Cl. CPC A61B 5/4851; A61B 5/6862; A61F 2/2418; A61F 2250/0002; A61B 2562/0247

(57) ABSTRACT

The present invention is directed to a system for monitoring the functional status of an implantable heart valve. The system includes wireless pressure sensors that are embedded in the implantable heart valve. An external device receives signal transmitted from the wireless pressure sensors. The external device reads and analyzes these signals and then transmits the data to a healthcare provider.

### CROSS REFERENCE TO RELATED ΔPPLICATIONS

This application claims the benefit of U.S. Provisional Patent Application No. 62/864,871 filed on Jun. 21, 2019, which is incorporated by reference, herein, in its entirety.

### GOVERNMENT SUPPORT

This invention was made with government support under CBET-1511200 awarded by the National Science Foundation. The government has certain rights in the invention.

### FIELD OF THE INVENTION

The present invention relates generally to medical devices. More particularly, the present invention relates to an embedded sensor system for wireless monitoring of the functional status of implanted heart valves.

### BACKGROUND OF THE INVENTION

Advances in sensor miniaturization, functional materials, telemetry and signal processing are opening up new opportunities for implantable wireless sensors to monitor organ function/health. A target of opportunity in this regard is the embedding of such sensors into prostheses or organ implants to provide longitudinal/persistent monitoring of prosthesis function and early detection of onset of potential adverse outcomes. Sensors embedded into or integrated within prosthetic valves have the advantage that this avoids the additional complication and risk of a separate medical procedure for implanting the sensor.

While a wide range of prosthetic devices/implants (orthopedic implants, breast implants, cerebrospinal shunts, etc.) could benefit from such monitoring, cardiac/cardiovascular implants/prostheses are one class of implants where such monitoring has high potential for decreasing adverse outcomes.

Due to increasing life expectancy combined with the advent of trans-catheter delivery of cardiovascular devices, the number of cardiovascular implants such as heart valves, rings, clips, and embolization protection devices is growing exponentially. Transcatheter heart valves in particularly are being deployed at extremely high rates projected to reach 850,000 heart valves/year by 2050.

The vast majority of these are transcatheter aortic valves (TAVs). These devices are expensive (approximately USD $32,000) and have a relatively complex design comprising of an expandable metal stent frame and bioprosthetic leaflets. Some new transcatheter valves are also employing polymeric or other synthetic materials (see FIG. 1).

The global Transcatheter Aortic Valve Replacement (TAVR) devices market size was valued at USD 2.4 billion in 2017 and global Transcatheter Aortic Valve Implantation (TAVI) procedures market size was valued at USD 6.2 billion in 2017. It is anticipated to rise at a CAGR of 22.6% over the forecast period. Rising number of patients across the globe suffering from cardiovascular diseases such as heart failures, coronary artery diseases, and hypertension is one of the key trends stoking market growth. Cardiovascular diseases lead to improper functioning of aortic valves, which in turn escalates the demand for efficient valve replacement procedure. In addition, increasing funding for research & development activities, new product launches, and presence of favorable reimbursement policies are some of the factors likely to provide a fillip to the market. Growing adoption of minimally invasive surgery (MIS), along with technological advancements in transcatheter aortic valve replacement procedure, is projected to reduce the overall hospital stay. This, in turn, is poised to reduce healthcare expenditure. Furthermore, geographic expansion, funding, and adoption of advanced devices & procedural methods are estimated to boost the growth of the market during the forecast period. According to Annals of Cardiothoracic Surgery, Transcatheter Aortic Valve Implantation (TAVI) devices are considered to be cost-effective in treating high risk patients. In European and American countries, TAVI devices are government funded.

Transcatheter aortic valves have a biomimetic design with three leaflets and optimal function of these valves usually corresponds to unimpeded and coordinated (in-phase) opening and closing of the three leaflets, see FIGS. 1 and 2A-2E. Transcatheter valves implanted in other locations (specifically, mitral, tricuspid and pulmonic) also have similar characteristics.

During implantation, the implanting surgeon/cardiologist must maneuver the implant to an optimal location in the aortic annulus and orient it appropriately in order to ensure that the prosthesis anchors into the aortic root and then functions with acceptable forward flow performance and little to no regurgitation. Peri-procedural imaging options for guiding implant placement are limited to echocardiography (transthoracic or transesophageal) and fluoroscopy. These modalities provide two-dimensional images and cannot accurately image the dynamic motion of each of the leaflets. Thus, the implanting surgeon cannot assess in real-time if the leaflets are opening fully or the degree of symmetry in the leaflet opening. Furthermore, the implanting surgeon cannot currently determine with accuracy, the azimuthal (angular) position of the TAV leaflets relative to the native leaflet and sinuses which is important for commissural alignment. Finally, the implanting surgeon cannot currently determine the position of the TAV leaflets relative to the coronary ostia nor the functional status of the TAV leaflets that are over the coronary ostia.

Accordingly, there is a need in the art for an embedded sensor system for wireless monitoring of the functional status of implanted heart valves.

### SUMMARY OF THE INVENTION

The foregoing needs are met, to a great extent, by the present invention which provides a system for monitoring the functional status of an implantable heart valve including an implantable heart valve. The system includes sensors positioned on the implantable heart valve. The sensors are configured for monitoring the functional status of the implantable heart valve. The sensors are also configured for outputting a signal, wirelessly, wherein the signal contains data related to the functional status of the implantable heart valve.

In accordance with an aspect of the present invention, the sensors are positioned along an inflow lumen of the implantable heart valve. The sensors are centered with respect to prosthetic leaflets of the implantable heart valve. The sensors are positioned on a frame of the implantable heart valve behind prosthetic leaflets of the implantable heart valve. Sensors can also be positioned on a distal edge of a frame of the implantable heart valve. The sensors can take the form of pressure sensors, accelerometers, and strain sensors. The sensors are configured to estimate individual motion of each of prosthetic leaflets of the implantable heart valve. The system includes an external device to receive the signal transmitted from the sensors. The system further includes a non-transitory computer readable medium programmed for analyzing the signal transmitted from the sensors and output information to a healthcare provider.

In accordance with an aspect of the present invention, the system includes a non-transitory computer readable medium programmed to process the assimilates of each of the sensor data such that the functional status of the heart valve, the heart, and the vasculature is determined. The system includes an external module to facilitate transmission of data to and from the sensors. The external module is further configured to transmit data to external locations, devices, or storage. The external locations, devices, or storage take the form of one selected from the group of a caregiver, server, a computing device, and medical devices or equipment.

In accordance with yet another aspect of the present invention, the sensors take the form of tandem pairs, wherein each tandem pair transmits data as a unit. The sensors are positioned circumferentially about the valve in two separate circumferential planes. In other embodiments the sensors are positioned circumferentially about the valve. The valve can include a stent. The sensors can also be positioned circumferentially about the stent. Alternately. the sensors are positioned on the frame of the implantable heart valve, in the sinuses of Valsalva.

### BRIEF DESCRIPTION OF THE DRAWING

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1 illustrates views of exemplary implantable heart valves.

FIGS. 2A-2E illustrate views of a self-expanding transcatheter aortic valve.

FIG. 3 illustrates image views of evidence of valve thrombosis and its effect on leaflet mobility.

FIGS. 4A and 4B illustrate perspective views of a sensor system where sensors are fixed on a supra-annular stent frame, near the sino-tubular junction, according to an embodiment of the present invention.

FIGS. 5A-5C illustrate perspective views of a sensor system where sensors are fixed on valve leaflets, according to an embodiment of the present invention.

FIGS. 6A-6C illustrate perspective views of a sensor system where sensors are fixed in a flow-facing portion of the sub-annular skirt, according to an embodiment of the present invention.

FIGS. 7A-7C illustrate perspective views of a sensor system where sensors are fixed in a tissue-facing region of the sub-annular skirt, according to an embodiment of the present invention.

FIGS. 8A and 8B illustrate perspective views of a sensor system where sensors are fixed on the stent frame, at the aortic sinus level, according to an embodiment of the present invention.

FIGS. 9A-9C illustrate perspective views of a set of six tandem sensors fixed on sub-annular and a supra-annular region of the heart valve.

FIGS. 10A-10C illustrate perspective views of a set of six tandem sensors fixed on the sinus and aortic (STJ) regions of the heart valve stent.

FIGS. 11A-11C illustrate flow and graphical views of computational fluid dynamics simulation results of transvalvular flows with normal and abnormal prosthetic aortic valves.

FIGS. 12A and 12B illustrate flow and graphical views of variation of pressure change due to the reduced leaflet motion.

FIGS. 12C-12E illustrate perspective and graphical views of in-vitro measurement with a pressure sensor on the supra-annular region of the TAV.

FIGS. 13A-13B illustrate graphical views of pressure signals from the tandem sensors for each leaflet. FIGS. 13C and 13D illustrate graphical views of leaflet motion presented by the distance from the valve center to the leaflet tips (Tip distance).

FIG. 14 illustrates graphical views of histograms of discrimination indices obtained from linear discrimination analysis (LDA) with the number of principal component analysis (PCA) modes chosen for optimal prediction using the sinus, aortic and tandem sensor configurations.

FIG. 15 illustrates graphical views of prediction of individual leaflet range of motion on the training and testing sets using the three sensor configurations, with PCA modes tuned for optimal prediction.

FIG. 16 illustrates a schematic view of an embedded sensor system for at home transcatheter valve monitoring, according to an embodiment of the present invention.

FIG. 17 illustrates a schematic view of an embedded sensor system for at-home transcatheter monitoring, according to an embodiment of the present invention.

FIG. 18 illustrates a schematic diagram of an embedded sensor system for a Cath-lab transcatheter valve positioning system.

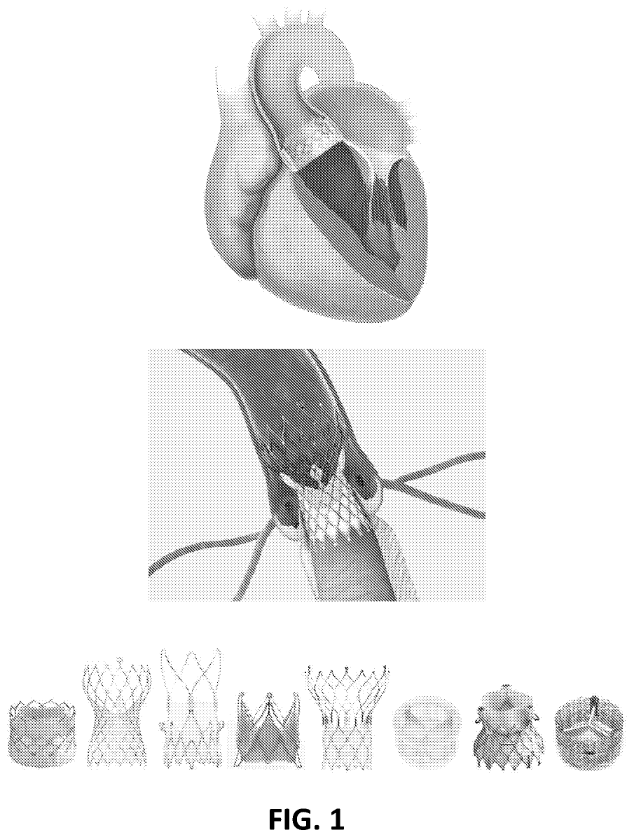

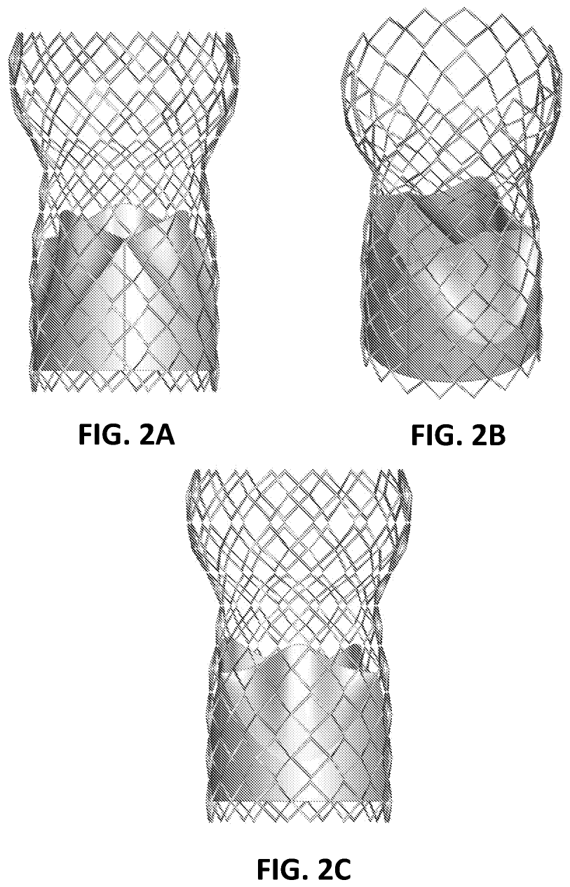

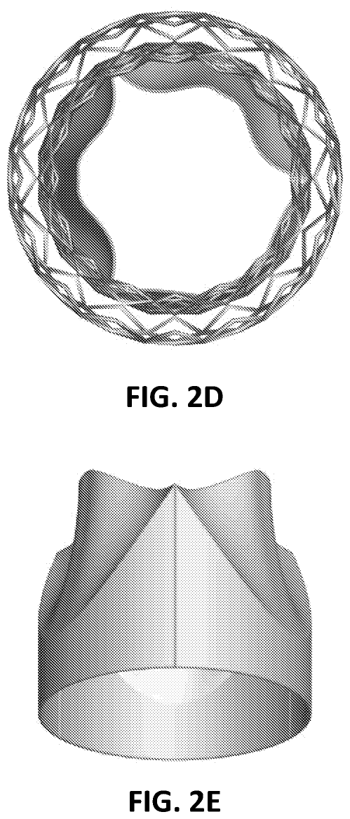

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims. It should be noted that any dimensions are included simply by way of example and are not meant to be considered limiting. Any suitable dimensions known to or conceivable to one of skill in the art could also be used.

The present invention is directed to a system for monitoring the functional status of an implanted heart valve. The system includes wireless pressure sensors that are embedded in the heart valve. An external device receives signal transmitted from the wireless pressure sensors. The external device reads and analyzes these signals and then transmits the data to a healthcare provider.

In contrast to the current state of the art, the current system incorporates optimal sensor placements that are not obvious. The sensors are positioned along the inflow lumen centered with respect to the prosthetic leaflets. Sensors are positioned behind the leaflets on the stent frame as well as along a distal edge of the stent frame. This placement plan for the sensors is novel and derived from computational and experimental studies to optimize the position. The dynamic nature of sensor activation with respect to set conditions is also novel. Furthermore, the use of the sensors to estimate individual leaflet motion to aid in delivery of the device is also novel. Lastly, the sensor configuration also enables the estimation of ventricular and vascular health status and is clearly distinct from current state of the art.

The wireless sensor system of the present invention includes sensors that are embedded in the TAV. The signal transmitted by the sensors, can be analyzed to identify anomalous TAV function and leaflet motion and would be extremely useful during the implantation procedure. Such a system would reduce reliance on imaging, which is either invasive (such as Transesophageal echocardiography) or exposes the patient to radiation (such as fluoroscopy), or does not provide three-dimensional information about the location and orientation of the implant and its leaflets.

Prior art in this arena (see US20070173861A1) employs pressure sensors mounted on the tip of catheters. Such sensors cannot communicate wirelessly with an external device. Simultaneous measurements from different locations cannot be made with this device and precise positioning of the pressure sensor with respect to the valve is not possible.

The direct exposure of heart valve prostheses to the dynamic flow of blood as well as the fact that they are in direct contact within the surrounding vascular or cardiac tissue (e.g. the aortic root or the mitral annulus) that undergoes movement and deformation during each cardiac cycle makes them prone to a variety of “malfunctions” such as leaflet/valve thrombosis, infections, paravalvular leaks, regurgitation, calcification and mechanical deformation. These malfunctions can remain clinically and symptomatically silently or occur catastrophically within minutes, both compromising the long-term health of the heart as well as patient's quality of life. Currently, there are no at-home monitoring systems for evaluating the functional status of implanted heart valves (transcatheter or surgical; aortic, mitral, tricuspid or pulmonic position).

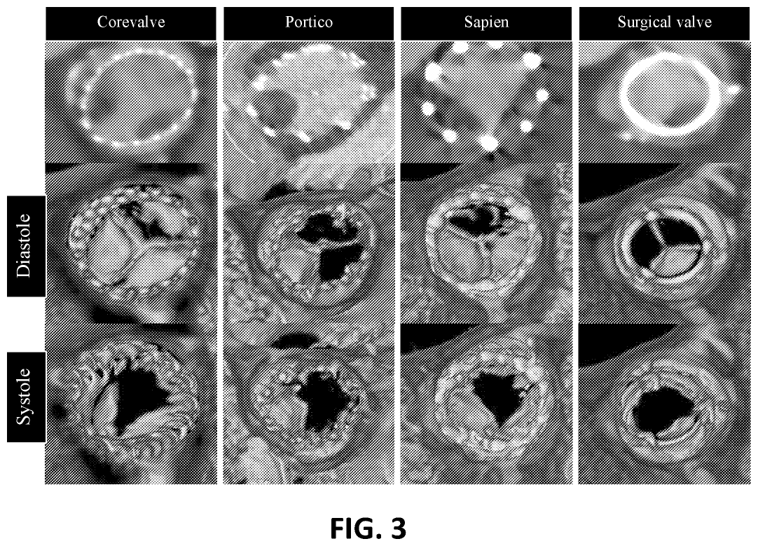

Among these various conditions, early leaflet thrombosis is one that has emerged as the most concerning. Recent studies have indicated that nearly all the TAVs on the market are susceptible to early leaflet thrombosis. Furthermore, this condition represents a major health-risk for the patient because while it is clinically “silent” (hemodynamic gradients are subclinical), it significantly increases the risk for thromboembolic events such as embolic stroke. Moreover, there is evidence that the risk of TAV thrombosis may persist beyond one year after valve implantation. This underscores the importance of serial follow-up and evaluation of the risks and benefits of long-term anticoagulation at regular intervals. Four-dimensional multi-detector computed tomography (4D-MDCT) is currently the most specific and sensitive imaging technique for detecting reduced leaflet motion (RLM) and leaflet thickening associated with leaflet thrombosis, see FIG. 3. However, persistent monitoring of TAV status and leaflet function in implanted TAVs using this type of hospital-centric imaging is cost-prohibitive and poses high exposure to ionizing radiation. Current standard-of-care typically requires that TAVR patients visit the implanting cardiologist at 1 and 12 months post-implantation, but no monitoring of the TAV implant is conducted in between these visits.

A wireless sensor system where the sensors are embedded in the TAV and the signal transmitted by the sensors can be analyzed to (a) monitor TAV performance; (b) identify anomalous TAV function and leaflet motion, and (c) monitor overall parameters related to cardiac function, and would be extremely useful for long-term, at-home monitoring of TAV functional status and the overall cardiovascular status of the patient.

A variety of implantable microsensors have been developed for in-situ monitoring of the hemodynamic status of the cardiovascular system (CardioMEMS sensor from Abbot (Brugts 2017), Cordella System from Endotronix, etc.). Many of these sensors are designed to sense pressure, and similar pressure sensors could be used in the current system. Other sensors such as accelerometers, strain sensors etc. could also be used in the sensor system. These sensor systems generally include an off-body external module that receives the signal from the sensor. This external module may also send a signal to the embedded sensors. This external module may be capable of transmitting data to other devices (computers, phones, tablets) or networks (Wi-Fi, cellular) via wired or wireless connections. This external module is also capable of analyzing and processing the signal with appropriate hardware (CPU etc.) and software.

Given the inherent temporal and spatial complexity of the pressure field within the aorta and the multivariate nature of the implantation procedure as well as conditions such as leaflet thrombosis, the use of more than one sensor will enhance detection and classification of these anomalies. However, sensors placed in “insensitive” or “nondiscriminatory” locations would increase the system complexity and cost, without enhancing its diagnostic capability. The number of sensors and their placement configuration on the TAV is therefore a key element of the system design which would be a function of the valve position (aortic, mitral, etc.), and stent design.

Advances in sensor miniaturization, functional materials, telemetry and signal processing are opening up new opportunities for implantable wireless sensors to monitor organ function/health. A target of opportunity in this regard is the embedding of such sensors into prostheses or organ implants to provide longitudinal/persistent monitoring of prosthesis function and early detection of onset of potential adverse outcomes. Sensors embedded into or integrated within prosthetic valves have the advantage that this avoids the additional complication and risk of a separate medical procedure for implanting the sensor.

While a wide range of prosthetic devices/implants (orthopedic implants, breast implants, cerebrospinal shunts, etc.) could benefit from such monitoring, cardiac/cardiovascular implants/prostheses are one class of implants where such monitoring has high potential for decreasing adverse outcomes.

Due to increasing life expectancy combined with the advent of trans-catheter delivery of cardiovascular devices, the number of cardiovascular implants such as heart valves, rings, clips, and embolization protection devices is growing exponentially. Transcatheter heart valves in particularly are being deployed at extremely high rates projected to reach 850,000 heart valves/year by 2050.

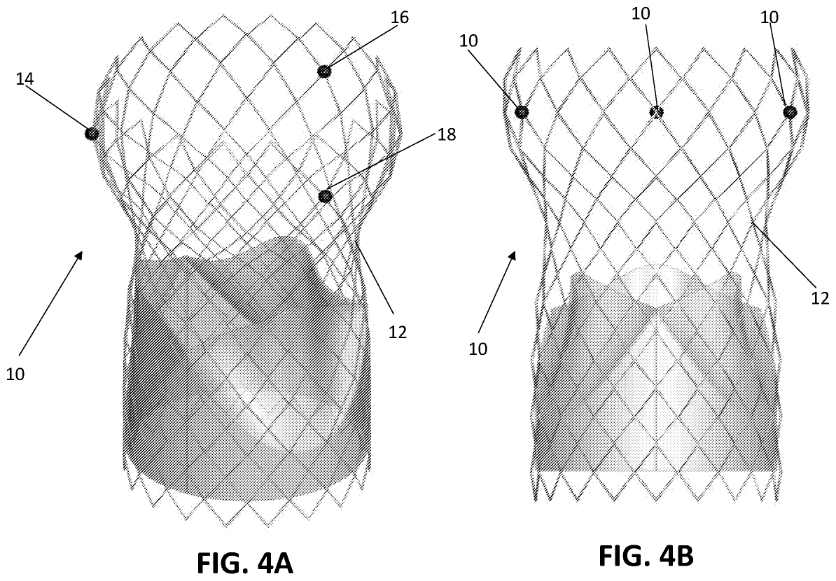

FIGS. 4A and 4B illustrate perspective views of a sensor system where sensors are fixed on a supra-annular stent frame, near the sino-tubular junction, according to an embodiment of the present invention. As illustrated in FIGS. 4A and 4B, the sensor system 10 is fixed on the supra-annular stent frame 12, near the aortic sino-tubular junction (STJ) region. The sensor system 10 includes a trio of sensors 14, 16, 18 disposed in the STJ region. The embodiment illustrated in FIGS. 4A and 4B shows three sensors, however any number of sensors known to or conceivable to one of skill in the art could also be used. Additionally, as illustrated the sensors 14, 16, and 18 of the sensor system 10 are positioned circumferentially about the stent frame 12. However, any positioning arrangement in the STJ region known to or conceivable to one of skill in the art could also be used.

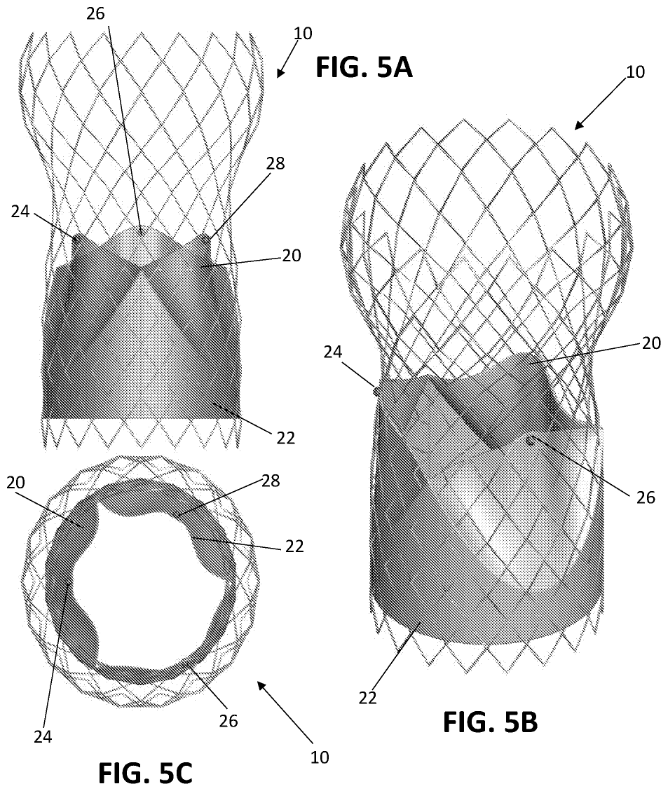

FIGS. 5A-5C illustrate perspective views of a sensor system where sensors are fixed on valve leaflets, according to an embodiment of the present invention. As illustrated in FIGS. 5A-5C, the sensor system 10 is positioned on leaflets 20 of the valve 22. The sensor system 10 includes sensors 24, 26, and 28 positioned on leaflets 20. The sensors 24, 26, 28 are positioned circumferentially on the leaflets, as illustrated in the embodiment shown in FIGS. 5A-5C. However, any positioning arrangement on the leaflets known to or conceivable to one of skill in the art could also be used.

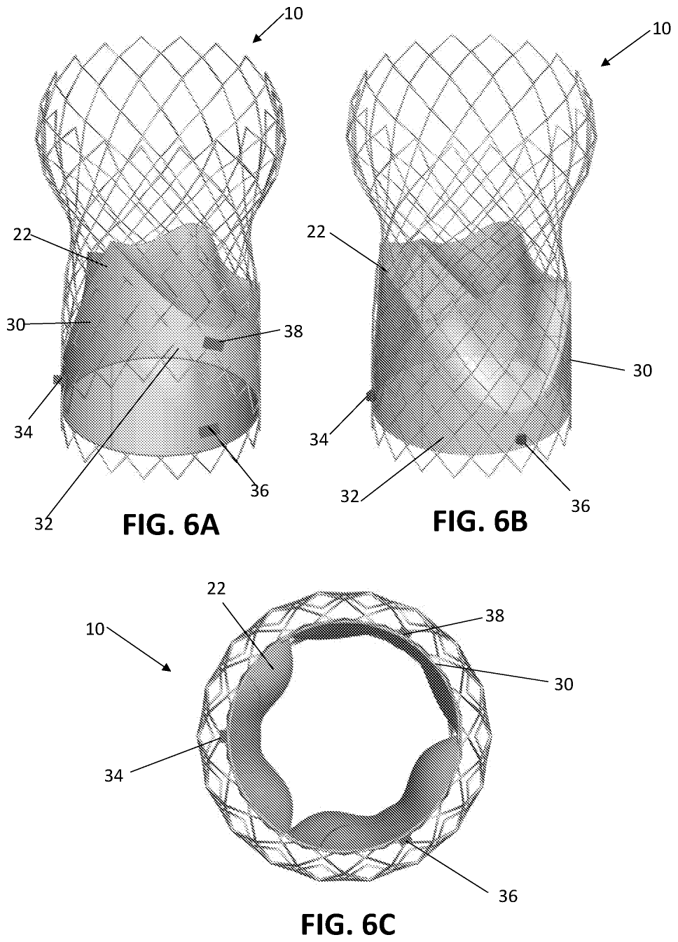

FIGS. 6A-6C illustrate perspective views of a sensor system where sensors are fixed in a flow-facing portion of the sub-annular skirt, according to an embodiment of the present invention. As illustrated in FIGS. 6A-6C, the sensor system 10 is positioned on the flow-facing side 30 of the subannular (skirt) region 32 of the valve 22. The sensor system 10 includes sensors 34, 36, and 38 positioned on valve 22. The sensors 34, 36, and 38 are positioned circumferentially about the skirt, as illustrated in the embodiment shown in FIGS. 6A-6C. However, any positioning arrangement on the flow-facing side 30 of the skirt region 32 known to or conceivable to one of skill in the art could also be used.

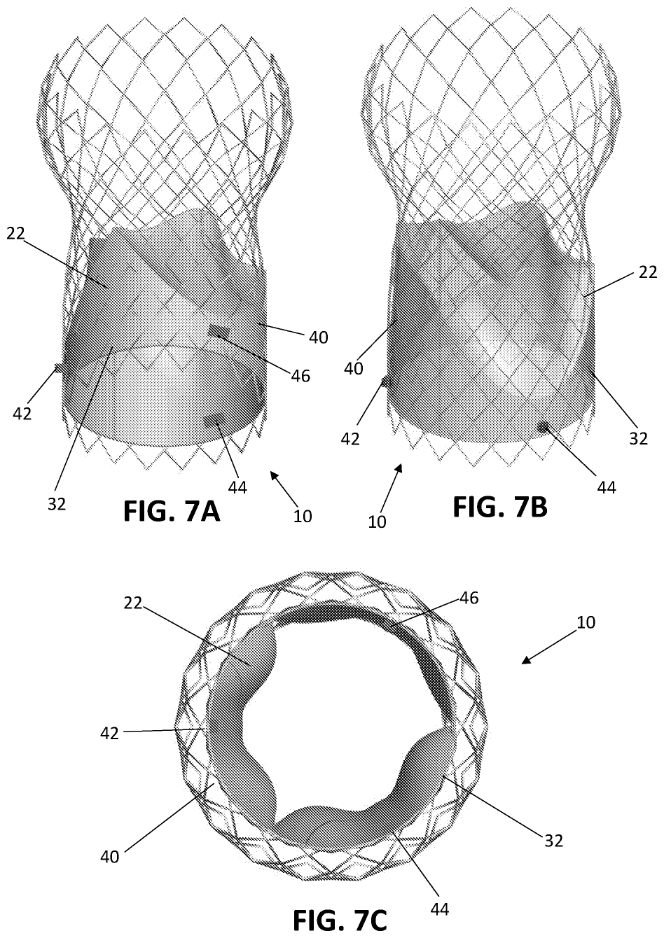

FIGS. 7A-7C illustrate perspective views of a sensor system where sensors are fixed in a tissue-facing region of the sub-annular skirt, according to an embodiment of the present invention. As illustrated in FIGS. 7A-7C, the sensor system 10 is positioned on the tissue-facing side 40 of the subannular (skirt) region 32 of the valve 22. The sensor system 10 includes sensors 42, 44, and 46 positioned on leaflets 20. The sensors 42, 44, and 46 are positioned circumferentially about the skirt, as illustrated in the embodiment shown in FIGS. 6A-6C. However, any positioning arrangement on the tissue-facing side 40 of the skirt region 32 known to or conceivable to one of skill in the art could also be used.

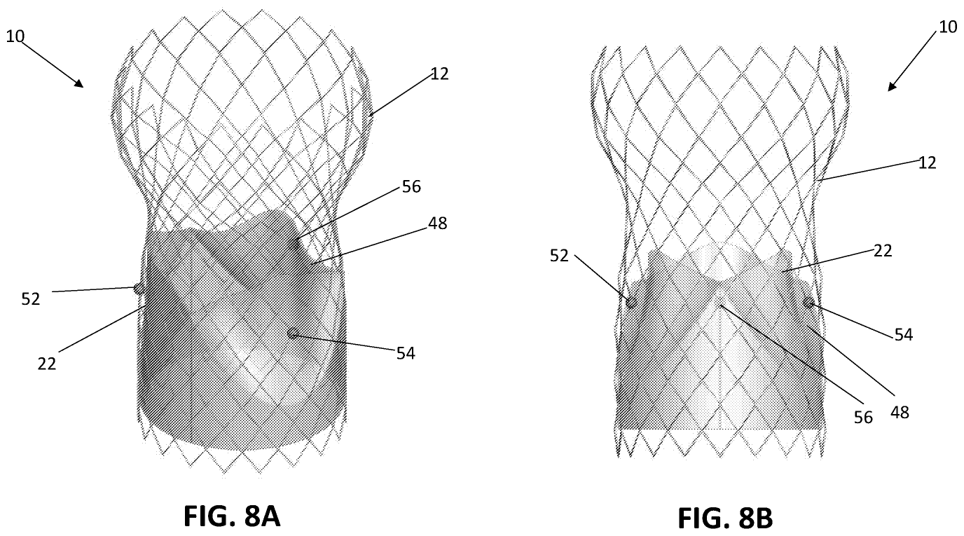

FIGS. 8A and 8B illustrate perspective views of a sensor system where sensors are fixed to the stent frame near cusps of the aortic sinus region of the stent, according to an embodiment of the present invention. As illustrated in FIGS. 8A and 8B the sensor system 10 is positioned on the stent frame 12 near cusps 48 on the aortic sinus region 50 of the valve 22. The sensor system 10 includes sensors 52, 54, 56 positioned near cusps 48. The sensors 52, 54, and 56 are positioned circumferentially near the cusps 48, as illustrated in the embodiment shown in FIGS. 8A and 8B. However, any positioning arrangement on the stent frame near the cusps known to or conceivable to one of skill in the art could also be used.

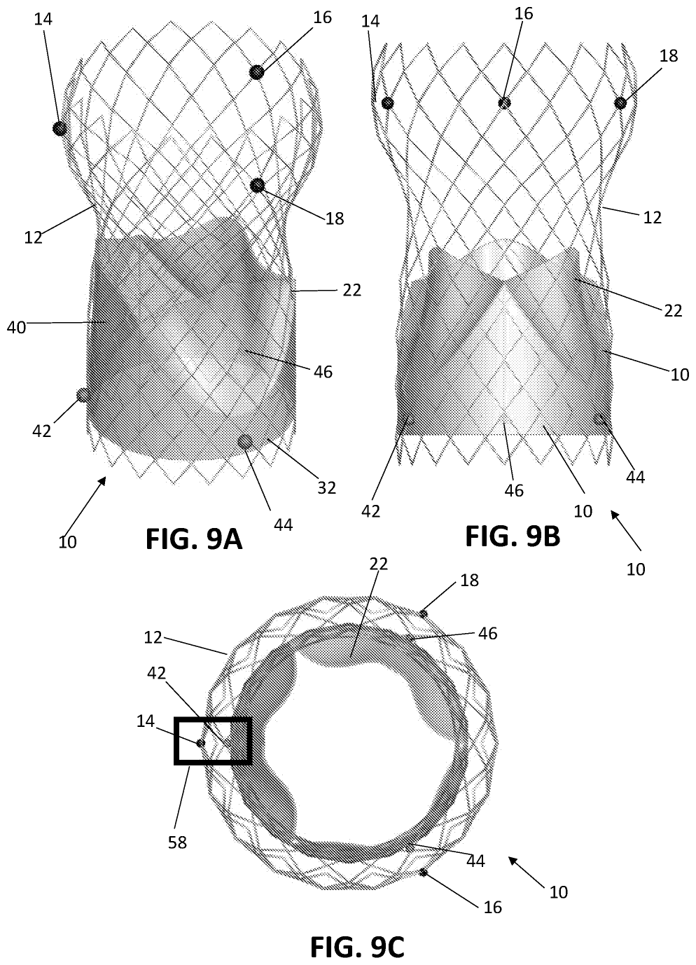

FIGS. 9A-9C illustrate perspective views of a set of six tandem sensors fixed on sub-annular and a supra-annular region of the heart valve. As illustrated in FIGS. 9A-9C, the sensor system 10 is fixed on the supra-annular stent frame 12, near the STJ region. The sensor system 10 includes a trio of sensors 14, 16, 18 disposed in the STJ region. The embodiment illustrated in FIGS. 9A-9C shows three sensors, however any number of sensors known to or conceivable to one of skill in the art could also be used. Additionally, as illustrated the sensors 14, 16, and 18 of the sensor system 10 are positioned circumferentially about the stent frame 12. However, any positioning arrangement in the STJ region known to or conceivable to one of skill in the art could also be used. Further as illustrated in FIGS. 9A-9C, the sensor system 10 is also positioned on the tissue-facing side 40 of the subannular (skirt) region 32 of the valve 22. The sensor system 10 further includes sensors 42, 44, and 46 positioned on the valve 22. The sensors 42, 44, and 46 are positioned circumferentially about the skirt, as illustrated in the embodiment shown in FIGS. 9A-9C. However, any positioning arrangement on the tissue-facing side 40 of the skirt region 32 known to or conceivable to one of skill in the art could also be used. Additionally, with respect to the embodiment shown in FIGS. 9A-9C the sensors 14, 16, and 18 are paired with sensors 42, 44, and 46 resperctively in tandem pairs 58. As illustrated in the embodiment of FIGS. 9A-9C there are three tandem pairs formed from the six sensors.

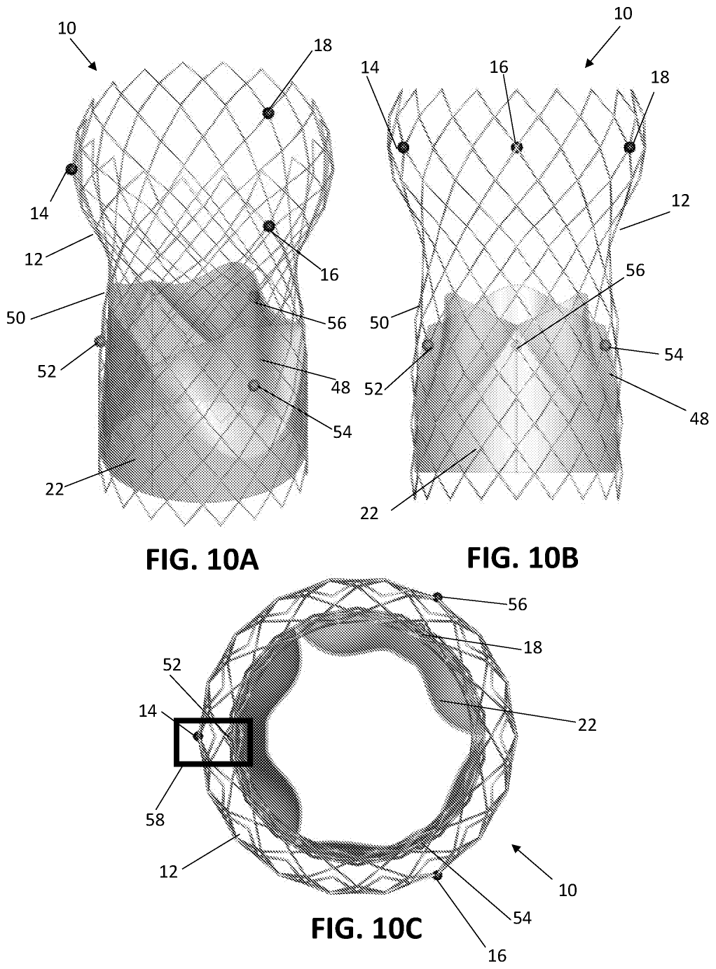

FIGS. 10A-10C illustrate perspective views of a set of six tandem sensors fixed on the sinus and aortic (STJ) regions of the heart valve stent. As illustrated in FIGS. 9A-9C, the sensor system 10 is fixed on the supra-annular stent frame 12, near the STJ region. The sensor system 10 includes a trio of sensors 14, 16, 18 disposed in the STJ region. The embodiment illustrated in FIGS. 10A-10C shows three sensors, however any number of sensors known to or conceivable to one of skill in the art could also be used. Additionally, as illustrated the sensors 14, 16, and 18 of the sensor system 10 are positioned circumferentially about the stent frame 12. However, any positioning arrangement in the STJ region known to or conceivable to one of skill in the art could also be used. Further as illustrated in FIGS. 10A-10C, the sensor system 10 is positioned on the stent frame 12 near cusps 48 on the aortic sinus region 50 of the valve 22. The sensor system 10 includes sensors 52, 54, 56 positioned on the stent frame 12 near cusps 48. The sensors 52, 54, and 56 are positioned circumferentially on the stent frame 12 near cusps 48, as illustrated in the embodiment shown in FIGS. 10A-10C. However, any positioning arrangement on the stent frame near the cusps known to or conceivable to one of skill in the art could also be used. Additionally, with respect to the embodiment shown in FIGS. 10A-10C the sensors 14, 16, and 18 are paired respectively with sensors 52, 54, and 56 in tandem pairs 58. As illustrated in the embodiment of FIGS. 10A-10C there are three tandem pairs formed from the six sensors.

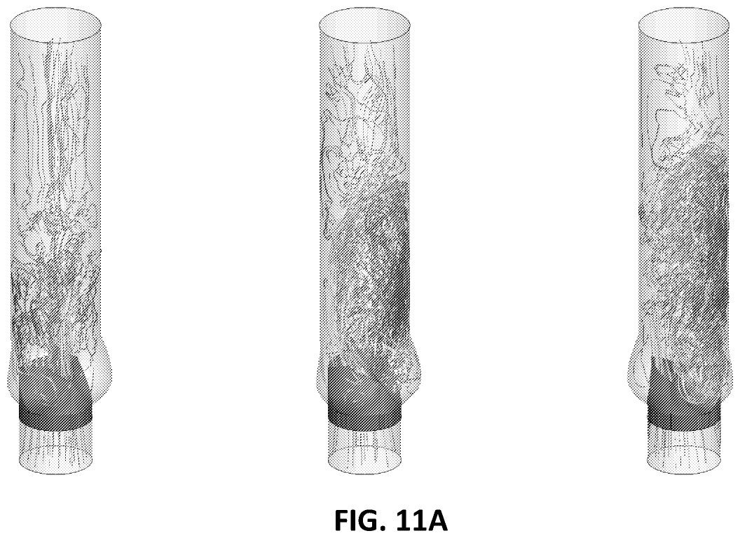

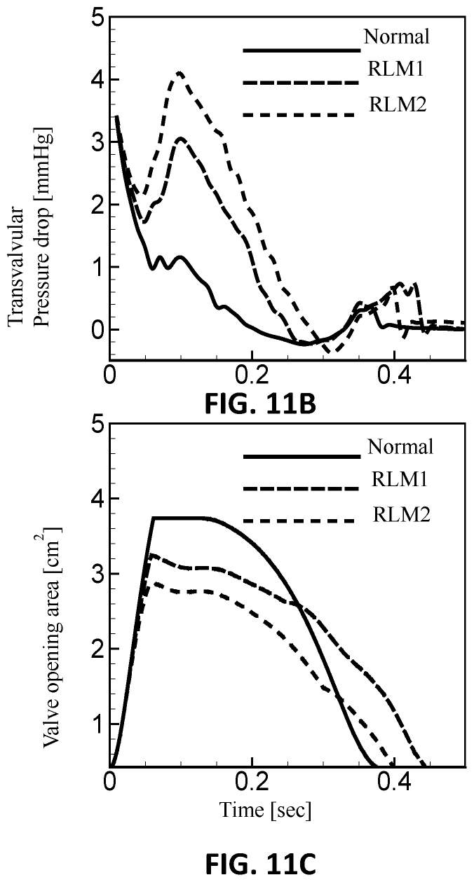

FIGS. 11A-11C illustrate flow and graphical views of computational fluid dynamics simulation results of transvalvular flows with normal and abnormal prosthetic aortic valves. FIG. 11A illustrates simulation views of instantaneous stream traces. FIG. 11B illustrates a graphical view of time histories of transvalvular pressure drop. FIG. 11C illustrates a graphical view of time histories of projected valve opening area. Normal: normal valve, RLM1: reduced leaflet motion on 1 leaflet due to increased stiffness, RLM2: reduced leaflet motion on 2 leaflets.

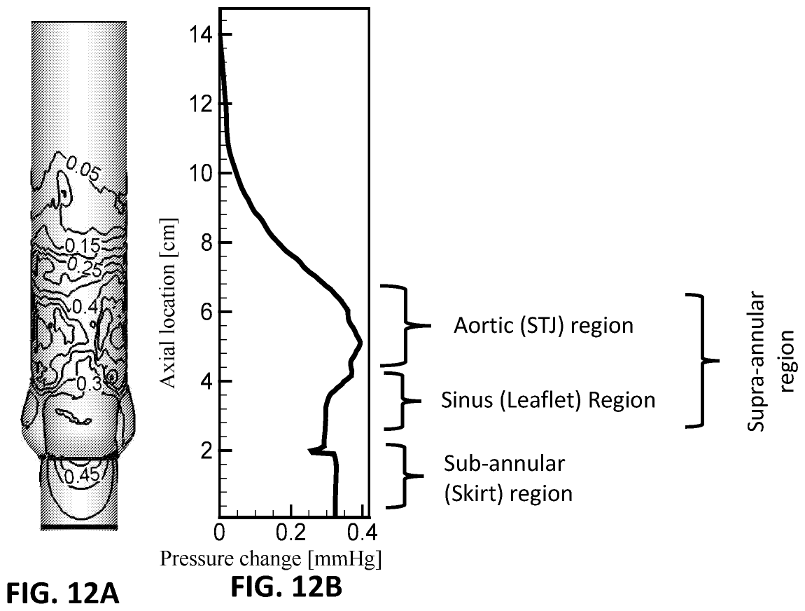

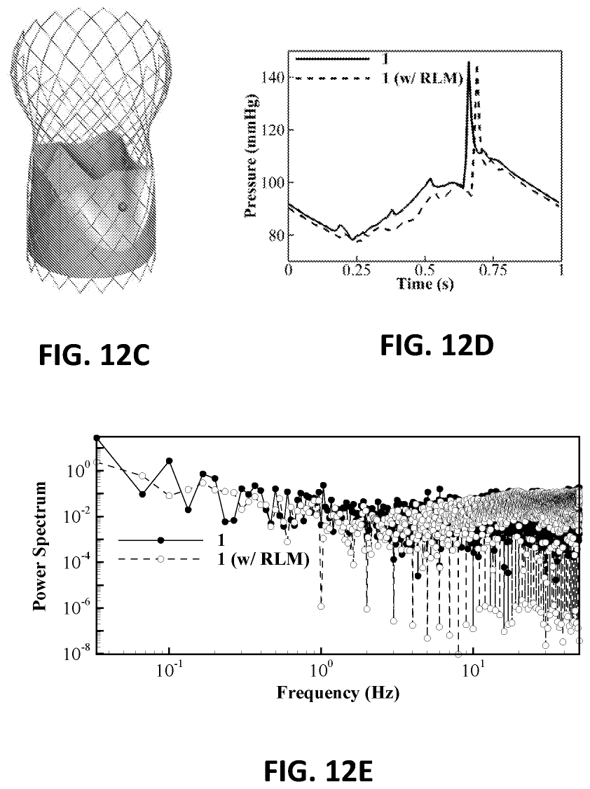

FIGS. 12A and 12B illustrate flow and graphical views of variation of pressure change due to the reduced leaflet motion. FIG. 12A illustrates a spatial map of the pressure change in [mmHg]. FIG. 12B illustrates a graphical view of axial variation of the circumferentially averaged pressure change.

FIGS. 12C-12E illustrate perspective and graphical views of in-vitro measurement with a pressure sensor on the supra-annular region of the TAV. FIG. 12C illustrates a perspective view of an exemplary sensor placement on the supra-annular region of the TAV. FIG. 12D illustrates a graphical view of a sensor signal for a normal valve and a valve with anomalous reduced leaflet motion. FIG. 12E illustrates a graphical view of a fourier transform of the sensor signals.

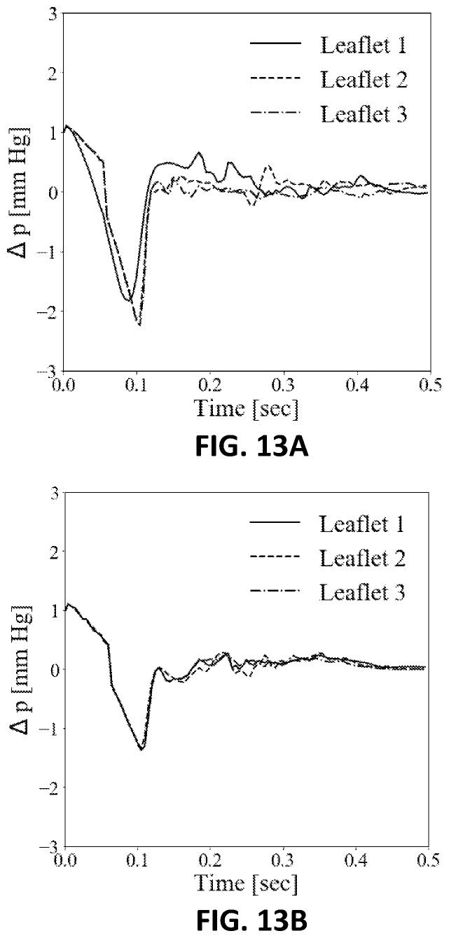

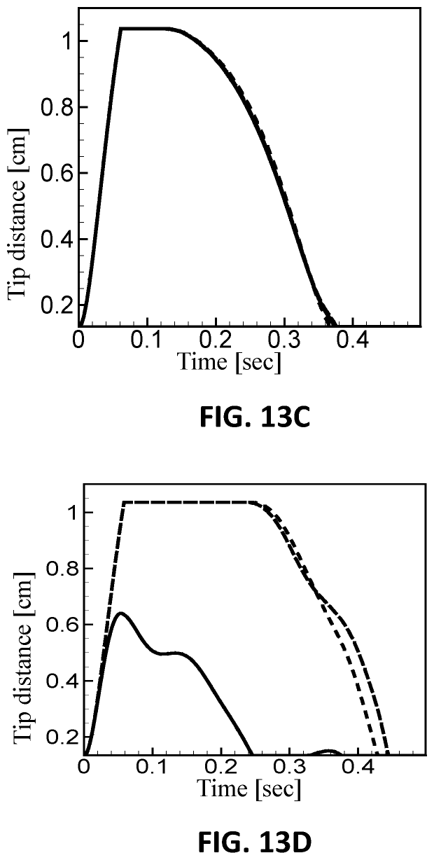

FIGS. 13A-13B illustrate graphical views of pressure signals from the tandem sensors for each leaflet. FIGS. 13C and 13D illustrate graphical views of leaflet motion presented by the distance from the valve center to the leaflet tips (Tip distance). FIGS. 13A and 13C represent data taken from a normal valve. FIGS. 13B and 13D represent data taken from a valve with reduced leaflet motion on 1 leaflet (Leaflet 1).

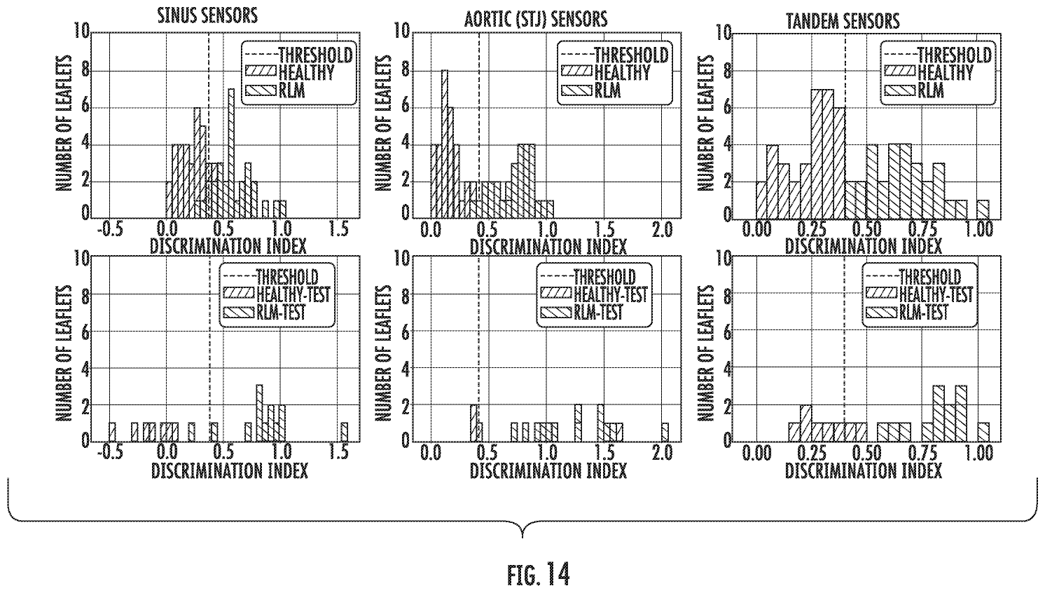

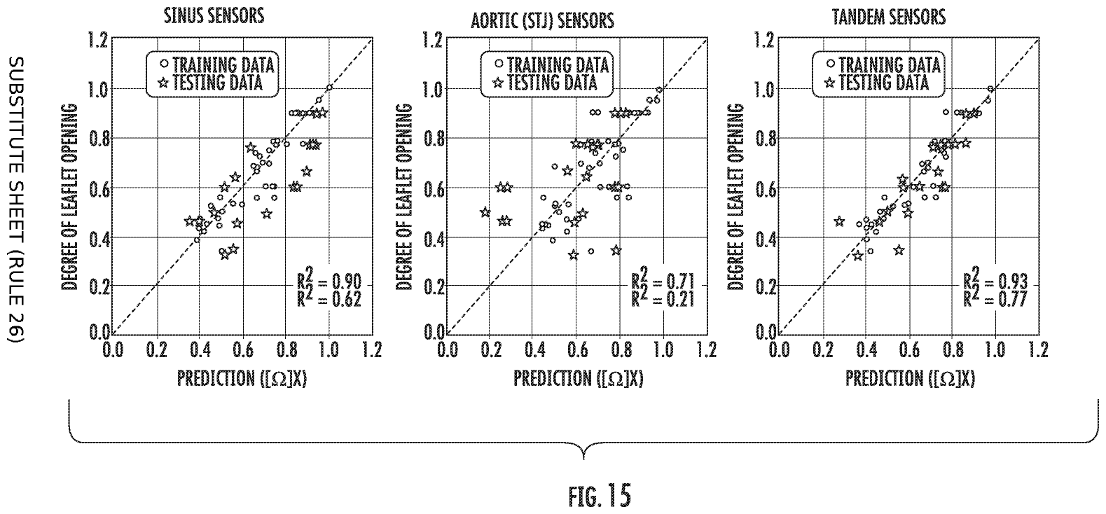

FIG. 14 illustrates graphical views of histograms of discrimination indices obtained from linear discrimination analysis (LDA) with the number of principal component analysis (PCA) modes chosen for optimal prediction using the sinus, aortic and tandem sensor configurations. Light shaded bars for normal cases and dark shaded bars for abnormal cases (Total 84 cases). FIG. 15 illustrates graphical views of prediction of individual leaflet range of motion on the training and testing sets using the three sensor configurations, with PCA modes tuned for optimal prediction.

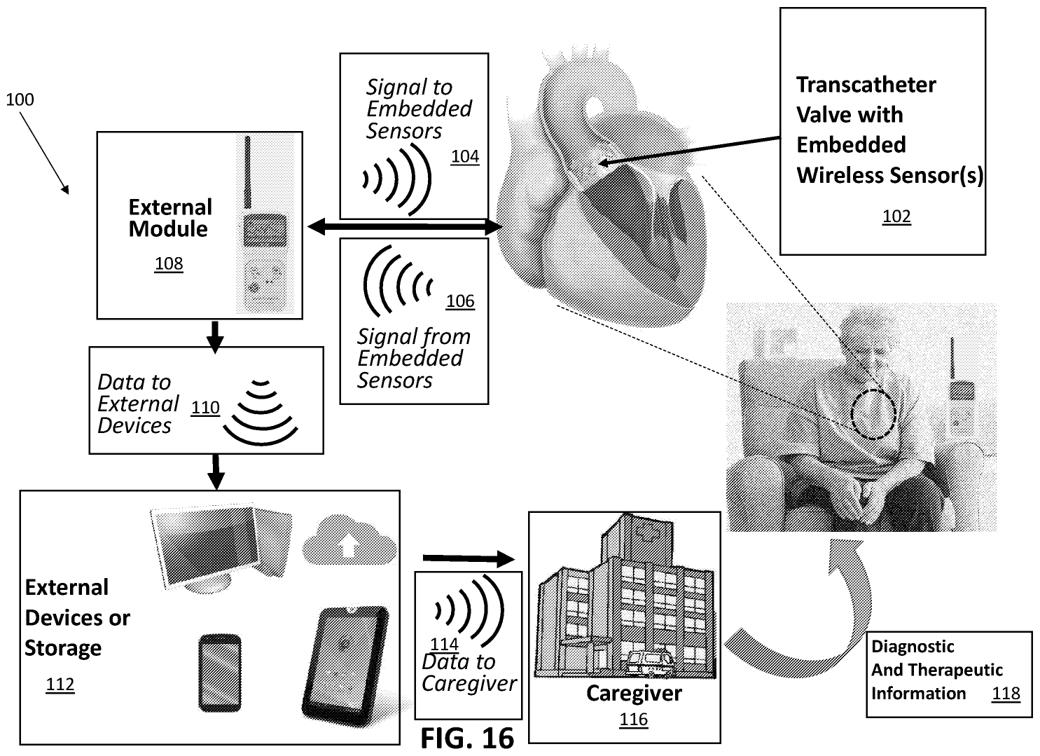

FIG. 16 illustrates a schematic view of an embedded sensor system for at home transcatheter valve monitoring, according to an embodiment of the present invention. The system 100 of FIG. 16 includes a transcatheter valve with embedded wireless sensors 102 according to the one of the embodiments described herein. The transcatheter valve with embedded wireless sensors 102 receives signal 104 to the embedded sensors from an external module 108. The embedded sensors also transmit signal to the external module 108. The external module can take the form of any computing device known to or conceivable to one of skill in the art including a specially designed console for the system 100. In order to make use of the data from the embedded sensors, the external module 108 transmits data 110 to external devices 112. External devices can take the form of any computing device known to or conceivable to one of skill in the art, such as a smartphone, tablet, laptop, device console, etc. Data 114 is then transmitted from the external devices or storage to a caregiver 116. The caregiver 116 can use the data to transmit diagnostic and therapeutic information and instructions to the patient.

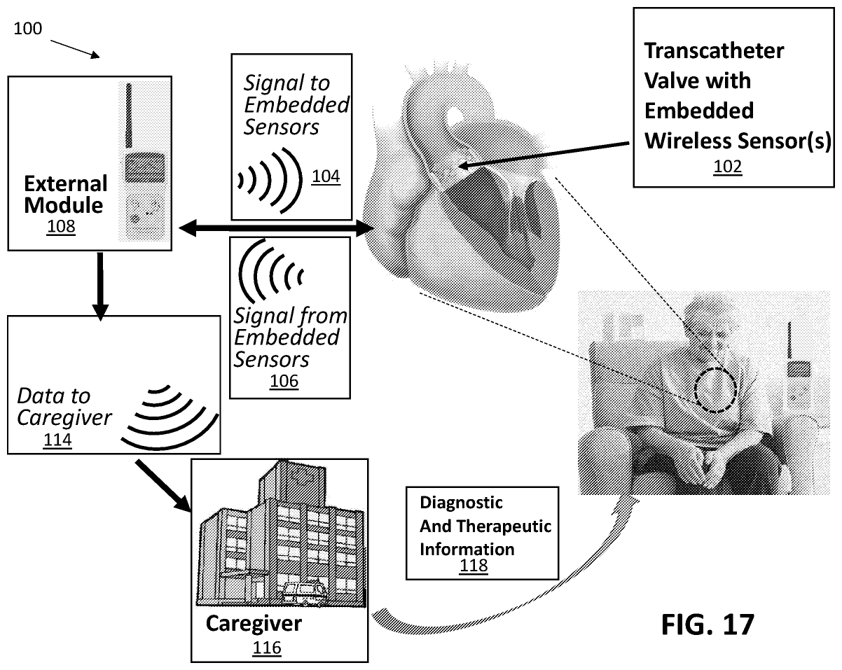

FIG. 17 illustrates a schematic view of an embedded sensor system for at-home transcatheter monitoring, according to an embodiment of the present invention. The system 100 of FIG. 17 includes a transcatheter valve with embedded wireless sensors 102 according to the one of the embodiments described herein. The transcatheter valve with embedded wireless sensors 102 receives signal 104 to the embedded sensors from an external module 108. The embedded sensors also transmit signal to the external module 108. In order to make use of the data from the embedded sensors, the external module 108 transmits data 114 to a caregiver 116. The caregiver 116 can use the data to transmit diagnostic and therapeutic information 118 and instructions to the patient.

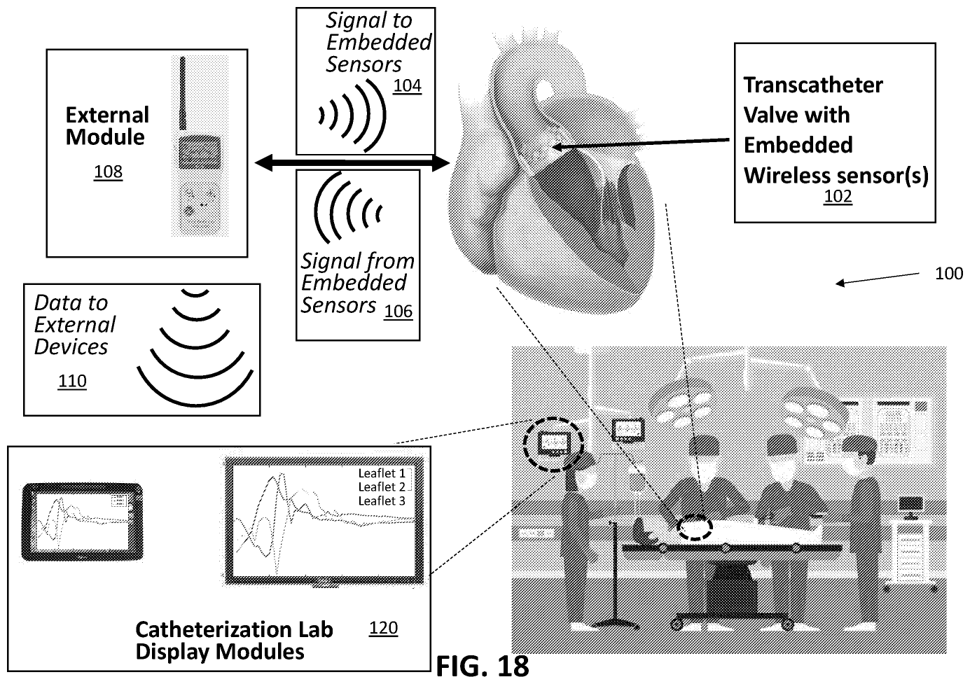

FIG. 18 illustrates a schematic diagram of an embedded sensor system for a Cath-lab transcatheter valve positioning system. The system 100 of FIG. 18 includes a transcatheter valve with embedded wireless sensors 102 according to the one of the embodiments described herein. The transcatheter valve with embedded wireless sensors 102 receives signal 104 to the embedded sensors from an external module 108. The embedded sensors also transmit signal to the external module 108. In order to make use of the data from the embedded sensors, the external module 108 transmits data 110 to external devices 120. Here, the external devices 120 are catheterization lab display modules.

As described above, sensors can be embedded (a) in the sub-annular (or skirt) region; (b) supra-annular portion of the implant, in the sinus region or near the sino-tubular junction (STJ); and (c) in or on either side of the leaflet, as illustrated in FIGS. 4-10. Studies in conjunction with the present invention indicate preferential locations for the placement of sensors which maximize the detectability of valve anomalies. Simulations of blood flow through bioprosthetic aortic valves without and with valve anomalies, as illustrated in FIGS. 11A-11C, show that one region where valve anomalies generate large changes in pressure is the inflow lumen of the valve where the skirt of the valve surrounds the metal stent frame, as illustrated in FIGS. 10A-10C. Thus, the skirt region of the valve is a preferential location for these sensors. The skirt region location is available for embedding of sensors for both balloon-expandable and self-expandable TAVs. The simulations also show that sensors that are aligned with the center of the valve leaflet provide the largest detectability of the leaflet anomalous motion.

The other location where valve anomalies generate large changes in pressure is downstream of the valve where the vortices are formed in the transvalvular jet and also where the transvalvular jet impacts the aortic wall, as illustrated in FIG. 12A. Simulations show that if a leaflet does not open in a normal way, the direction of the jet is altered, and a sensor located in a region downstream of the valve opening can pick up the change in pressure due to this flow behavior. Thus, the supra-annular region of the valve is a preferential location for these sensors.

Locations on the stent in the aortic sinus behind the mobile prosthetic leaflets are also regions where valve anomalies cause large pressure changes. This region is largely exposed to recirculating flow behind the mobile leaflet. In each cusp of the aortic sinus, the flow is relatively agnostic to that in the other two. Thus, measurements form this region are highly sensitive to changes in valve function. FIG. 12B shows in-vitro measurement from a sensor placed in the sinus region of the TAV.

Another location where valve anomalies generate large changes in pressure is on the valve leaflets, as illustrated in FIGS. 10A-10C. Thus, the leaflets of the valve are a preferential location for these sensors. Studies associated with the present invention also indicate that sensors configured in a tandem configuration such that one sensor is embedded in the sinus region and the other sensor is directly above the leaflet attachment on the stent, near the sino-tubular junction, as illustrated in FIGS. 10A-10C is a particularly effective configuration for determining the function of the leaflets and the implant. When placed in this configuration, the following methods of signal analysis can be employed: subtracting the signal from these two sensors provides a direct measure of the pressure drop between the sinus and the aorta. If the signal from the sinus sensor is P1(t) and the signal from the aortic sensor is P2(t), then the instantaneous pressure drop across the sinus is

ΔP(t)=P1(t)−P2(t)

where ‘t’ refers to time.

Likewise, the instantaneous pressure drop across the valve (referred to as the transvalvular gradient) may be computed as the difference between measurements upstream (at the skirt location) and downstream (at the STJ) from the valve. A large value of pressure drop, averaged over systole,(greater than about 10 mm of mercury) usually indicates non-negligible flow blockage most commonly due to reduced geometric opening of the leaflets. This is an important measure of overall valve function and performance. However, the sinus pressure difference is found to be more sensitive to changes in individual leaflet mobility and can capture changes in corresponding leaflet function.

FIG. 13 shows the ΔP signal versus leaflet opening from one of our simulation studies.

Tandem sensors can be embedded for each leaflet and the signals from these tandem sensor arrangements can be analyzed to identify leaflets that are opening and moving normally from leaflets that are experiencing anomalous movement. FIG. 14-15 shows a series of analyses carried out using the ΔP(t) from the tandem sensor configuration.

These sensor arrangements can indicate the position of paravalvular leaks when comparing ΔP(t) during the period the valve is closed. The sensor data can also estimate the patient's real time ventricular as well as arterial properties. For example, the time-rate-of-change of pressure at the inflow region of the valve can be correlated to the stroke volume and contractility of the ventricle. The changes in pressure (such as the decay constant) during the period the valve is closed can estimate the structural compliance of the aorta (for the case of the aortic valve). These parameters can be derived over resting and moderate activity of the patient to gauge changes in exercise physiology of the patient such as the responses to natural vasodilation as well as surrogate pressure volume characteristics of the ventricle. Complete characterization of the ventricular function will help manage heart failure therapy for the patient if the patient developed heart failure prior to or after valve replacement. The pressure rise over the iso-volumetric contraction phase can be calculated based on comparing the pressure changes between the inflow and outflow regions of the valve. This parameter gives a direct measure of myocardial contractility.

Sensors imbedded in or attached to the valve leaflets provide a strong signal of anomalous movement of the leaflet. The signal from the sensors in this case combines the effects of pressure as well as leaflet acceleration. The acquisition of the signal by the external module and the processing of the sensed signal is tightly coupled with the number and placement of sensors and is therefore an integral part of the system design. A sophisticated sensor system that is coupled with an inappropriate signal acquisition or signal analysis framework (and vice-versa) would diminish the effectiveness of the system.

The signal acquisition protocol could be controlled by the external module; a controller inside the external module, or a connected device, could modify the data acquisition protocol “on-the-fly” based on the real-time analysis of the signal. The analysis of the signal could involve comparison with previously obtained signals as well as other patient data acquired previously. Changes in sensor data acquisition include but are not limited to changing the temporal rate of signal acquisition from one or more sensors, changing signal intensity to one or more sensors, not acquiring signals from one or more sensors, increasing the duration of the signal acquisition from one or more sensors, and increasing the rate at which the patient is prompted to make these measurements during a given period.

The following are some example scenarios where such “on-the-fly” changes would be employed by the sensor system in the setting of post-operative monitoring of the implant (see FIGS. 16 & 17):
The signal from the embedded sensor(s) is analyzed by the external module to determine the heart-rate. If the heart rate is outside the range considered nominal for the patient, the external module stops the signal acquisition and signals the patient to acquire the measurement at a later time.
The module determines from the signal that the heart rate outside a pre-programmed range and changes the signal acquisition and/or analysis based on programmed criteria.
The external module determines the signal-to-noise ratio in the signal (this noise may be, for instance, due to the external environment or excessive movement by the patient) and based on this, either changes the signal acquisition or stops the signal acquisition and signals the patient to acquire the measurement at a later time.
The external module determines from the signal that one or more of the sensors are malfunctioning and changes the signal acquisition and/or the method of signal analysis based on programmed criteria.
The analysis of the signal indicates that the valve or leaflet is not functioning normally. The external module then modifies the signal acquisition and/or signal analysis based on programmed criteria.
Programmed criteria may be based on new calculations to estimate the extent of severity of the anomaly such as estimating leakage volume fraction, energy loss, extent of thrombosis (mass, or volume), leaflet stiffness, etc.

The following are some example scenarios where such “on-the-fly” changes would be employed by the sensor system in the setting of peri-procedural guidance, as illustrated in FIG. 18 during the implantation procedure:
The analysis of the signal from the embedded sensor(s) indicates that the valve or one or more leaflets are not functioning normally. The external module then modifies (automatically or based on input from the surgeon) the signal acquisition and/or signal analysis based on programmed criteria to provide additional data to the implanting surgeon/cardiologist.
Additional data may include corrective steps to reposition the valve in specific directions.
Additional data may include real-time rendering of signal inferred leaflet opening/closing.
Once the surgeon finalizes the placement of the implant, he/she instructs the external module to enter a “baselining” mode where it continuously acquires the signal and establishes the baseline signal that is later used for diagnostic purposes. In this “baselining” mode, the external module follows a signal acquisition sequence where it acquires signals over a large range of conditions (heart rates, time-of-day, sleeping, awake, etc.) of the patient. This sequence requires the external module to determine the heart rate and other measures from the signal, and then modify the signal acquisition accordingly.
Analysis of the signal could be carried out by the external module and/or by other devices to which the external module can send and receive data such as computers, cell phones and other such devices. The signal analysis framework would extract the key features of the signal and generate metrics that quantify or correlate with TAV and leaflet function.
Finally, transmission of the raw and/or processed signal onto a device (computer, laptop, tablet or phone) and display of the processed information in the form of tables, plots, charts and others, that provides the cardiologist or caregiver the most accurate evaluation of leaflet anomalies is a key element of the system design.

The present invention could be carried out using a computer, non-transitory computer readable medium, or alternately a computing device or non-transitory computer readable medium incorporated into a console for TAV implantation. Indeed, any suitable method of calculation known to or conceivable by one of skill in the art could be used. It should also be noted that to the extent specific equations are detailed herein, variations on these equations can also be derived, and this application includes any such equation known to or conceivable by one of skill in the art.

A non-transitory computer readable medium is understood to mean any article of manufacture that can be read by a computer. Such non-transitory computer readable media includes, but is not limited to, magnetic media, such as a floppy disk, flexible disk, hard disk, reel-to-reel tape, cartridge tape, cassette tape or cards, optical media such as CD-ROM, writable compact disc, magneto-optical media in disc, tape or card form, and paper media, such as punched cards and paper tape. The computing device can be a special computer designed specifically for this purpose. The computing device can be unique to the present invention and designed specifically to carry out the method of the present invention. It is not a standard business or personal computer that can be purchased at a local store. Additionally, this computer carries out communications with the TAV devices through the execution of proprietary custom built software that is designed and written by the manufacturer for the computer hardware to specifically operate the scanner hardware.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1. A system for monitoring the functional status of an implantable heart valve, comprising:
an implantable heart valve; and
sensors positioned on the implantable heart valve and wherein the sensors are configured for monitoring the functional status of the implantable heart valve and the heart and wherein the sensors are configured for outputting a signal, wirelessly, and wherein the signal contains data related to the functional status of the implantable heart valve.

2. The system of claim 1 further comprising the sensors being positioned along an inflow lumen of the implantable heart valve.

3. The system of claim 2 further comprising the sensors being centered with respect to prosthetic leaflets of the implantable heart valve.

4. The system of claim 1 wherein the sensors are positioned on a frame behind prosthetic leaflets of the implantable heart valve.

5. The system of claim 1 wherein the sensors are positioned on a distal edge of a frame of the implantable heart valve.

6. The system of claim 1 wherein the sensors are positioned on the frame of the implantable heart valve, in the sinuses of Valsalva.

7. The system of claim 1 wherein the sensors comprise pressure sensors.

8. The system of claim 1 wherein the sensors comprise at least one selected from a group consisting of pressure sensors, accelerometers, and strain sensors.

9. The system of claim 3 wherein the sensors are configured to estimate individual motion of each of the prosthetic leaflets of the implantable heart valve.

10. The system of claim 1 further comprising an external device to receive the signal transmitted from the sensors.

11. The system of claim 10 further comprising a non-transitory computer readable medium programmed for analyzing the signal transmitted from the sensors and output information to a healthcare provider.

12. The system of claim 1 further comprising a non-transitory computer readable medium programmed to process the assimilates of each of the sensor data such that the functional status of the heart valve, the heart, and the vasculature is determined.

13. The system of claim 1 further comprising an external module to facilitate transmission of data to and from the sensors.

14. The system of claim 13 wherein the external module is further configured to transmit data to external locations, devices, or storage.

15. The system of claim 14 wherein the external locations, devices, or storage comprise one selected from the group consisting of a caregiver, server, a computing device, and medical devices or equipment.

16. The system of claim 1 wherein the sensors comprise tandem pairs, wherein each tandem pair transmits data as a unit.

17. The system of claim 16 wherein the sensors are positioned circumferentially about the valve in two separate circumferential planes.

18. The system of claim 1 wherein the sensors are positioned circumferentially about the valve.

19. The system of claim 1 wherein the valve further comprises a stent.

20. The system of claim 1 wherein the sensors are positioned circumferentially about the stent.
